(19) United States
(12) Patent Application Publication
(10) Pub. No.: US 2009/0047730 A1
(43) Pub. Date: Feb. 19, 2009

(54) Methods for Controlling Dust and Creating Bio-Crust

(76) Inventors: Timothy R. Higgins, Marana, AZ (US); Robert L. Redpath, Tucson, AZ (US)

Correspondence Address: QUARLES & BRADY LLP, ONE SOUTH CHURCH AVENUE, SUITE 1700, TUCSON, AZ, 85701-1621, US

(21) Appl. No.: 12/257,298

(22) Filed: Oct. 23, 2008

Related U.S. Application Data
(63) Continuation of application No. 11/681,091, filed on Mar. 1, 2007, now abandoned.

(51) Int. Cl. C12N 1/12

(52) U.S. Cl. 435/257.1

(57) ABSTRACT

Methods for controlling dust and other particulates using photosynthetic organisms to create a bio-crust. The methods include applying algae to a site under conditions suitable for forming a layer over the site that provides dust control.

### BACKGROUND OF THE INVENTION

1. Field of the Invention

The invention relates to a method that is especially useful for treating a mine tailing impoundment to prevent dust, and, more generally, to a method that utilizes photosynthetic organisms to provide an adhesive layer on a site, thereby preventing the distribution of particles through the creation of a layer or bio-crust.

2. Description of the Related Art

Milling and concentrating non-ferrous metals from ore material mined through open pit and underground mining methods removes most of the metals and generates sterile crushed rock of uniform size having little or no organic value. This crushed rock, commonly known as “mine tailings,” normally is deposited in surface impoundments or tailing dams. Tailings generally contain metals in a bound, non-extractable form and further contain a significant degree of moisture (up to 15%).

Tailing dams or impoundments can range in size—from several acres to thousands of acre—and can be up to hundreds of feet high. Generally, the tailings are deposited on or near the mine site in plain view, which tends to create aesthetic problems. Moreover, the tailing dams create environmental concerns generally related to dust and water erosion problems. In particular, because the mine tailings constitute very fine rock particles, wind can create the possibility of “dust storms” as the particles are distributed away from the intended site.

Conventional dust-control measures include applying magnesium chloride or other surface application compounds and/or applying crushed rock to the tailing piles. Rock coverings generally consist of applying a layer of crushed rock over the top of the tailing dams to prevent dust erosion of the tailings. The crushed-rock covering technique is intended for non-active tailings impoundments, to support vegetation to control erosion of the tailings, to influence water infiltration into the tailing dams, to provide a medium for plant root penetration, and to provide mechanical strength for the long term. However, unless a substantial amount of rock is applied, and generally reapplied over time, the application tends to be nothing more than a temporary solution. Tailing dams also tend to contain and hold water, something like a sponge, for very long periods of time.

Magnesium chloride is applied as a solution over the tailings. Magnesium chloride treatment is the process of combining magnesium chloride and water into a sprayable mixture and spraying that mixture onto the dust-generating tailings to induce crust development to keep the dust generating tailings in place. However, this method can be problematic and short-lived, depending on the environmental conditions at a particular site.

Hence, it would be useful to have a simple and inexpensive method for reliably controlling dust and creating a bio-crust over surfaces such as mine tailings.

### SUMMARY OF THE INVENTION

The invention generally involves methods for controlling dust and creating an adhesive layer of material, such as a bio-crust, that is generated from photosynthetic organisms. More particularly, the method generally relates to a process for creating a productive bio-crust on essentially worthless waste lands, such as those containing mine tailings, as well as other generally damaged or disturbed areas. The invention has been tested on a tailing structure located at an active copper mine in Arizona.

In one embodiment, the invention utilizes algae and includes analyzing a site for dust-control suitability, mathematically determining the bio reactor volume (e.g., organism supply pond size) needed to produce photosynthetic organisms (i.e., algae), mathematically determining the amount of growth nutrients to be applied to the bio reactor, mathematically determining algae flow volume from the bio reactor to the site, supplying the bio reactor with water with a continuous flow inlet valve and adding nutrients daily until the desired algae concentration is reached, and transporting algae from bio reactor to site for dispersion leading to generation of a bio-crust. The system of this embodiment is a continuous flow operation that produces algae for use in dust control. Simply put, algae is fed daily and then transported as a solution to the site for bio-crust formation. Therefore, continuous input and output of water into the bio reactor must exist along with daily feeding.

In accordance with the invention, another embodiment involves a method for controlling particle distribution on a surface using at least one photosynthetic organism, wherein the organism is algae and the method includes applying a predetermined mixture of algae to the surface such that deposits (e.g., bio-crust) form on the particles of the surface, thereby limiting the distribution of those particles by wind and other forces.

Additional features and advantages of the invention will be forthcoming from the following detailed description of certain specific embodiments when read in conjunction with the accompanying drawings.

### BRIEF DESCRIPTION OF THE DRAWINGS

FIG. 1 is a flow diagram outlining an embodiment of the invention suited for a large-scale tailings pond.

FIG. 2 is a flow diagram of a preferred methodology in accordance with the present invention.

### DESCRIPTION OF THE PREFERRED EMBODIMENTS

The invention relates to method involving the dispersion of photosynthetic organisms to control dust, preferably through the formation of a bio-crust.

Algae are photosynthetic organisms that have a relatively simple structure compared to vascular plants. Algae are nearly ubiquitous, and may grow in waters of varying salinity, in intertidal regions, in soils, on buildings, living symbiotically within other organisms, or even on desiccated sand and rocks in deserts and on the snowy slopes of mountains. Limnologists are most concerned with phytoplankton, which may live in the open water or on mud and surfaces on the bottom. The presence of different types of pigments is one main characteristic used to classify the different types of algae. All algae contain chlorophyll a, along with other accessory pigments. The accessory pigments absorb different wavelengths of light, allowing the algae to capture more of the sun's energy for use in photosynthesis.

The three major groups of algae are known as “supergroups:” Archaeplastida, Cabozoa, and Chromalveolata. Another classification of algae is as Divisions (equivalent to Phyla in animals): Cyanophyta, Chlorophyta, Cryptophyta, Chrysophyta, Pyrrophyta, Bacillariophyta, Euglenophyta and Rhodophyta.

All members of Division Cyanophyta, the “blue-green algae,” are prokaryotic cells with no organized nucleus and no mitochondria or chloroplasts. Members of all other algal divisions are eukaryotic. Blue green algae contain phycobilins (phycoerythrin or phycocyanin) as accessory pigments to chlorophyll a. It is the presence of the blue pigment phycocyanin in many Cyanophyta that gives them their characteristic blue-green color. Some blue-greens can fix molecular nitrogen and are often responsible for the “pond scum” on eutrophic lakes.

Division Chlorophyta consists of the “green algae.” These phytoplankton have chlorophylls a, b and sometimes carotenoids. This is a very diverse division and typically about half of the species of phytoplankton in a lake will be chlorophytes. Much evidence, including similarities in pigments, suggests that vascular plants arose from algae of the Division Chlorophyta.

The presence of chlorophylls a and c characterizes members of the Division Cryptophyta. These phytoplankton are often unicellular and motile and are common in the Laurentian Great Lakes. Some members of this division are mixotrophic, meaning they can take up organic compounds in addition to producing them through photosynthesis. The chrysophytes, or golden-brown algae, contain chlorophyll a, β-carotene, and sometimes chlorophyll c. They are often flagellated and many are mixotrophic. Some members have siliceous scales or cysts. Members of the Division Pyrrophyta, also known as dinoflagellates, contain chlorophylls a and c, and may be armored with plates known as theca. They are strong swimmers and have two flagella in grooves. Dinoflagellates are responsible for toxic red tides in oceans and estuaries. The diatoms, members of the Division Bacillariophyta, contain chlorophylls a and c and xanthophylls. They have an external covering, or frustule, made of SiO₂. There are two major orders of diatoms, the centric diatoms, which have radial symmetry, and the pennate diatoms, which have bilateral symmetry. Diatoms are often important components of spring blooms.

There are a few planktonic members of the Division Euglenophyta. They contain chlorophylls a and b and are flagellated unicells with no cell wall. Similarly, there are only a few freshwater representatives of the Division Rhodophyta, or “red algae.” They are predominantly colonial, have chlorophylls a and c, β-carotene and some xanthophylls. Members of the Division Rhodophyta never have flagella or flagellated gametes.

Of all the types of photosynthetic organisms discussed above, laboratory analysis indicates over time that bio-crust formation is most associated with blue green and green algae. The dominance of blue green and green algae is determined by the pH of the bio reactor solution and the site material to be treated. The pH should be between 6 and 10 for best bio-crust formation. With the correct pH, blue green and green algae dominance varies with temperature. During winter months blue green is dominant while during summer months green is dominant.

In the context of certain embodiments of the present invention, bio-crust development is believed to result from a variety of factors given a variety of different site compositions. It should be appreciated that virtually any troubled mining environment may be treated to enhance bio-crust development. However, as previously noted, a particularly advantageous site composition with which the present invention may be employed comprises active mine tailing dams with active slurry flow. The present invention will therefore be described in that context. The skilled artisan, however, will readily appreciate a variety of other applications for use of the subject invention, only some of which are listed herein, and which are within the scope of this invention.

In the context of active mine tailing ponds or dams, milling practices reduce thousands of tons of solid rock into tiny particles each day. Under normal conditions, climate (water availability) is generally an important factor for bio-crust development. However, in tailing dams, water is abundant, and, thus, a trivial element to the process. The problem is that the mine tailings typically do not contain the organic or living matter necessary to sustain algae growth and bio-crust development.

As will be discussed in greater detail hereinbelow, a preferred method, in accordance with the present invention, generally provides the preferred photosynthetic organisms (e.g., blue green and green algae) to the site to promote bio-crust development. More particularly, the method of the present invention provides nutrients in the proper parts-per-million rates to encourage photosynthetic organism concentration and ultimately bio-crust development. Photosynthetic organism movement about the site tends to encourage mixing of the nutrients with the site materials, creating the needed mixture for growth and bio-crust development. In accordance with this methodology, the surface is stabilized and thus provides the necessary “dust-controlled” environment.

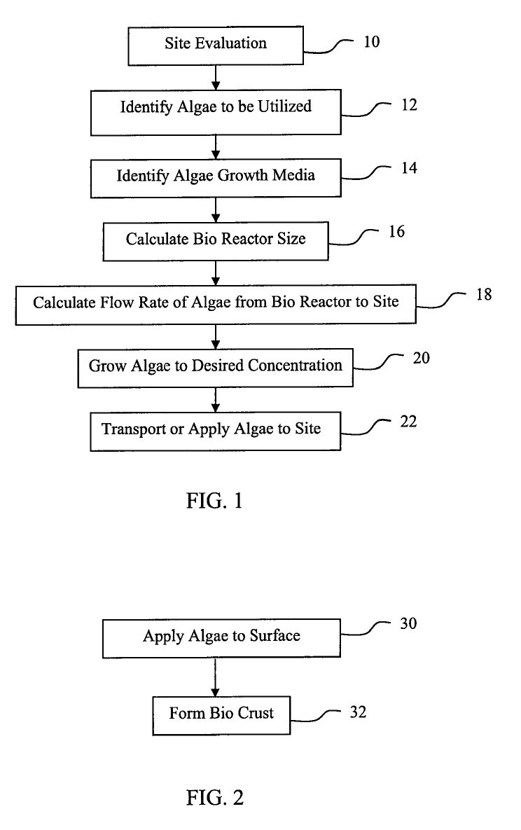

Referring now to FIG. 1, in accordance with an embodiment of the present invention suited for a large-scale tailings pond or dam, the method generally includes a first step 10 of analyzing or evaluating a site for organic and inorganic composition, a second step 12 of determining the type of algae to by used at the site, a third step 14 of determining the growth media or nutrients to be used in conjunction with the algae, the further step 16 of calculating a bio reactor size commensurate with the amount of algae needed for the site, the step 18 of calculating the flow rate of algae from the bio reactor to the site, the step 20 of growing the algae to the desired concentration in the bio reactor, and the step 22 of transporting or applying the algae to the site.

Preferably, in accordance with analysis step 10, the composition and the slope of the ground at the site is determined. More particularly, the ground may be analyzed for texture, density, percent sand, percent clay, percent silt, and moisture content, as well as any other ground or soil tests that may be pertinent in determining the composition and nutritional value of the ground. As will be appreciated, such analysis is conducted in accordance with conventional processes. As will be further appreciated, through analyzing the site, the particular composition of materials is determined. As noted hereinabove, various site compositions may be treated in accordance with the present invention, and the particular manner of treatment depends in large part on the site composition analysis. For a typical mine tailing dam, the site will have sufficient water to support algae growth but will not have sufficient nutrients available.

In accordance with a further aspect of analysis step 10, a site analyst may survey the site to determine its contour and slope (also in accordance with conventional techniques), such that the analyst may determine the amount of dust erosion that may occur if the site is left untreated. The site analyst further may determine the amount of algae-containing liquid slurry necessary to flow to the site for complete site coverage. If complete coverage cannot be obtained, dust generation may occur during actual treatment. The flow volume of the transport mechanism (e.g., a pump) along with the contour of the site is needed for optimal photosynthetic organism coverage of the site. In some cases, site management may be necessary to obtain complete site coverage and otherwise to obtain effective bio-crust formation and particulate or “dust” control (such as through nutrient and pH adjustment).

In accordance with step 12, the type of photosynthetic organisms that are to be used at a site is determined. Generally speaking, it has been found that blue green and green algae perform well in a dust control application given a wide variety of water and weather conditions. For example, blue green is preferred during winter months, while green algae is preferred in summer months. In order to insure that the culture contains the correct type or types of algae, laboratory analysis or field identification using of a flurometer may be performed.

In accordance with nutrient determination step 14, the type and amount of nutrient to be applied is established. To induce photosynthetic organism growth, nutrients and light are generally needed. In accordance with a preferred aspect of the present invention, photosynthetic organisms are applied to the site with the application of the nutrients, and thus, the amount of photosynthetic organisms applied to the site tends to be a determination of the active gallons per minute from slurry flow. Alternatively, a site may first be “seeded” with nutrients, with the organisms being added afterward, either alone or with additional nutrients.

In accordance with step 16, the appropriate growth chamber, “pond,” or bio reactor size is determined, along with the amount of nutrients supplied to the growth chamber to enable algae growth to a particular concentration. For example:

$${Pond}_{size} = \frac{Cycle Time in Minutes \times AGPM}{{RET}_{c}}$$

### Where;

P_{S} is the bio reactor size in gallons,
AGPM is the active gallons per minute of photosynthetic organism discharged from slurry lines onto the site, and
RET_{c} is the constant used to determine bio reactor size in gallons.

Thus, if an open bioreactor needs to be cycled every six days (8640 minutes) to prevent mosquito growth, the AGPM is determined to be 12, and the RET constant is 0.5, the reactor size equals 207,360 gallons.

The amount of nutrients to be used preferably is determined by following the manufacture's directions. For example, for every 70 gallons of water within the bio reactor, 1 gallon of nutrient(s) is needed such that a bio reactor of approximately 210,000 gallons will require 3000 gallons of feed each week of operation or 429 gallons daily. The manufactured ingredients preferably should include, but are not limited to, phosphorus, iron, sulfur, fish solubles, seaweed extract, complex sugars, micronutrients, collodial phosphates and humic acid. Many suppliers produce products capable of growing algaes, e.g., the products sold under the brands Grow Power® and Sunburst Products® Age Old Bloom.

For mining operations, dust generators, such as tailings dams, are very large, e.g., 300 feet tall 20 miles wide, with a dust generating surface of 26 acres. Therefore, the slurry line that transports tailings from the mill to the dam site can be used as the algae concentrate transport mechanism. Slurry line flow, a high volume of crushed rock and water e.g. 100,000 gpm. Since the photosynthetic organisms are incorporated into the slurry flow, an equation must be used to determine photosynthetic organism flow from the bio reactor to the transport mechanism in gallons per minute (gpm). Thus, in accordance with step 18, the flow rate of algae to the site of dust-control treatment is calculated.

Testing indicates the optimal amount of algae for injection from the bio reactor into the slurry line is 16 gallons per minute. In fact, 5, 8, 10, 12 and 16 gpm were tested, and 16 gpm produced sufficient viable culture (some is lost due to slurry components that are toxic to algae) at the end of the discharge tubes leading onto the tailings dam. Dividing 16 gpm by a 50,000 gpm slurry flow rate is 0.00032 (Slurry Flow Constant). Multiplying the slurry flow constant 0.00032 with any slurry flow and bio reactor flow in gpm will be obtained. For example:

Actual bio reactor flow=0.00032 (Slurry Flow Constant)×50,000 gpm=16 gpm

Actual bio reactor flow=16 gpm×0.75 (75% successful flow rate)=12 gpm

Before flow from the bio reactor to the site can begin, the bio reactor must be inoculated with the desired algae and fed daily for up to about 21 days. In accordance with step 20, applying the nutrients at the calculated rate on a daily basis will produce a preferred concentration of 2×10⁶ cells per ml of algae. During this time, the bio reactor should be cycled continuously to avoid stagnant water. To avoid mosquitoes in the bio reactor, the entire volume of the bio reactor must cycle every 6 days, as the literature indicates mosquitoes develop from larvae to adult in 7 days.

A flurometer is capable of identifying blue green and green algae concentrations. The range of pH for optimal algae growth (blue green and green) is 6.5 to 10. Lower pH's produce toxic photosynthetic organisms, and a pH higher than 10 will alter growth conditions where photosynthetic organisms typically cannot survive. Now, in accordance with step 22, transport of the algae to the site may begin and site conditions monitored such that the production of a coating or bio-crust is effected.

For example, in many bodies of water, phosphorus is the least available nutrient, so its abundance—or scarcity—controls the extent of algae growth. Therefore, it may be desirable to supplement a site with one or more nutrients for algae. If more phosphorus is added to the water body from sewage treatment plants, urban or farmland runoff, lawn or garden fertilizers, septic tanks or other watershed or outside resources, or even if it is released from phosphorus-rich bottom sediments from plant die-off, more algae will grow. A simple test can be performed to measure the phosphorus concentration or nitrogen levels to determine if this is a problem for a particular site. Another important parameter to check is pH. Because the methods of the invention operate optimally between pH levels of 6-10, a buffering compound may be added to a site to keep growth conditions within an optimal pH for a given algae.

As depicted in FIG. 2, the method of the invention more broadly involves the step 30 of applying algae to a surface at a site 30 and the step 32 of growing photosynthetic organisms such that a bio-crust is produced. In other words, the algae produce a layer that provides an adhesive effect such that the transport of particles by wind and other forces is reduced. In smaller scale applications, algae in a growth media may be applied directly to a site. The site may be supplemented with additional moisture, buffering agents, and/or nutrients, depending on the site composition, such that the formation of a bio-crust occurs.

Photosynthetic organisms generally are present in native soil and in the air from the surrounding environment. On their own, these photosynthetic organisms cannot survive on mine tailings or other surfaces. However, in accordance with the present invention, an environment suitable for development of a bio-crust is advantageously obtained with the provided nutrients. As the photosynthetic organisms grow, they produce by-products of polysaccharide sugars which act as binding agents that attach to silicates. This process actually forms a thin layer or bio-crust preventing dust generation from climatic activity.

The inventors have found that when the method of the present invention is applied to mine tailings, for example, mine tailing dams, bio-crust can be formed relative quickly. For example, wind gusts of up to 68 mph have been recorded with no significance of dust generation. In a quite surprising and unexpected manner, after the process of the present invention has been performed on such tailings, the algae forms a firm barrier indicating binding of the tailings surface has occurred, all within days-to-weeks after commencement. This tends to illustrate that sand and other particles will be bound by the algae deposits to form a bio-crust.

In accordance with one aspect of the present invention, the nutrients can be any type of mineral or organic suitable for photosynthetic organism development. Moreover, under some circumstances and in certain locations, specially formulated supplemental nutrients may be used to help further develop slow growing photosynthetic organisms. This will help maintain the proper nutrient levels during use on the site.

It should be noted that the type and quantity of nutrients supplied to the photosynthetic organisms may depend on, among other things, the desired concentration of photosynthetic organisms, the site climatic conditions as determined in site analysis step 10, and the amount of slurry to be flowed onto the site (where relevant, such as for transporting algae to a tailings dam.

Various modifications are possible within the meaning and range of equivalence of the appended claims.

We claim:

1. A method for controlling particle distribution, comprising the step of applying algae to a surface under conditions suitable for said algae to produce a deposit on the surface that inhibits particle movement.

2. The method of claim 1, wherein said surface comprises mine tailings.

3. The method of claim 1, wherein said suitable conditions comprise a pH of between 6 and 10.

4. The method of claim 3, wherein said algae is selected from the group consisting of green algae and blue green algae.

5. The method of claim 1, further including the step of providing at least one nutrient in mixture with said algae.

6. The method of claim 1, further including the step of providing at least one nutrient for algae to said surface.

7. The method of claim 1, wherein said deposit comprises a bio-crust.

8. The method of claim 1, wherein the algae is grown to an amount of between 1×10⁶ cells per ml and 1×10⁷ cells per ml prior to site application.

9. A method for controlling dust with algae, comprising the steps of:
(a) evaluating site conditions;
(b) growing a desired algae based on said site conditions; and
(c) applying said algae to said site such that a bio-crust forms.

10. The method of claim 9, wherein said site comprises mine tailings.

11. The method of claim 9, further including the step of calculating a size of a vessel used for growing said algae in step (b).

12. The method of claim 9, further including the step of calculating a flow rate of algae from a growth vessel to said site.

13. The method of claim 9, wherein the algae is grown to an amount of between 1×10⁶ cells per ml and 1×10⁷ cells per ml prior to step (c).

14. The method of claim 9, wherein algae growth is performed at a pH of between 6 and 10.

15. The method of claim 9, wherein said algae is selected from the group consisting of green algae and blue green algae.

16. The method of claim 9, further including the step of providing at least one nutrient in mixture with said algae.

17. The method of claim 9, further including the step of providing at least one nutrient for algae to said site.

18. The method of claim 9, further including adding a buffering compound to said site such that site pH is between 6 and 10.
